(19) United States
(12) Patent Application Publication
(10) Pub. No.: US 2008/0019907 A1
(43) Pub. Date: Jan. 24, 2008

(54) Optical Imaging Contrast Agents

(76) Inventors: Jo Klaveness, Oslo (NO); Edvin Johannesen, Oslo (NO); Helge Tolleshaug, Oslo (NO)

Correspondence Address: GE HEALTHCARE, INC., IP DEPARTMENT, 101 CARNEGIE CENTER, PRINCETON, NJ, 08540-6231, US

(21) Appl. No.: 10/582,680

(22) PCT Filed: Dec. 17, 2004
(86) PCT No.: PCT/NO04/00395

§ 371 Date: Apr. 16, 2007

(30) Foreign Application Priority Data
Dec. 19, 2003 (NO) 20035748

(51) Int. Cl. A61K 49/00

(52) U.S. Cl. 424/9.1

(57) ABSTRACT

The invention provides contrast agents for optical imaging of vulnerable atherosclerotic plaque in patients. The contrast agents may be used in diagnosis of vulnerable atherosclerotic plaque, for follow up of progress in disease development, for follow up of treatment of vulnerable atherosclerotic plaque and for surgical guidance. Further, the invention provides methods for optical imaging of vulnerable atherosclerotic plaque in patients.

### FIELD OF THE INVENTION

The present invention provides contrast agents for optical imaging of vulnerable atherosclerotic plaque in patients. The contrast agents may be used in diagnosis of vulnerable atherosclerotic plaque, for follow up of progress in disease development, and for follow up of treatment of vulnerable atherosclerotic plaque.

The present invention also provides new methods of optical imaging of vulnerable atherosclerotic plaque in patients, for diagnosis and for follow up of disease development and treatment of vulnerable atherosclerotic plaque.

### DESCRIPTION OF RELATED ART

Cardiovascular diseases kill about 15 million people in the world each year. Several of these die suddenly of a first myocardial infarction or cardiac arrest without any symptoms or diagnosis of coronary artery disease. Very many of these sudden deaths are caused by unstable or vulnerable plaque that suddenly blocks blood flow in critical arteries in the brain, the lungs or the heart. The rupture of vulnerable plaques contributes to about 75% of all myocardial infarctions and strokes. Today, no general diagnostic method is available for detection or characterisation of such plaques, but several methods have been suggested in the prior art. These vulnerable plaques consist of a lipid core (free and esterified cholesterol), macrophages, collagen and other matrix proteins.

Several methods have been suggested for detection of vulnerable atherosclerotic plaque. Some of these are drawn to techniques based on measurement of temperature. See e.g. U.S. Pat. No. 6,615,071 (The University of Texas) which suggests to detect vulnerable atherosclerotic plaque based on identification of regions with elevated temperature. U.S. Pat. No. 6,579,243 (SciMed Life Systems) describes a catheter with thermal sensor for detection of vulnerable plaque.

Other methods are directed to in vitro diagnosis based on samples from a patient. See e.g. U.S. Pat. No. 6,524,795 (Interleukin Genetics) which relates to diagnosis of plaque based on a nucleic sample from the patient and detection of IL-1 genotype patterns.

U.S. Pat. No. 6,375,925 (University of California) suggests non-invasive imaging of atherosclerotic plaque using labelled monoclonal antibodies which bind oxidation specific epitopes like oxidized LDL.

Further methods have been described using different diagnostic imaging modalities, but without use of a contrast agent. See e.g. U.S. Pat. No. 6,262,575 (Siemens) which describes a method of MR imaging of plaque identifying fat. No contrast agents are involved. U.S. Pat. No. 5,217,456 (PDT Cardiovascular) describes a method to differentiate healthy tissue from atherosclerotic plaque based on fluorescence signals. This is an intravascular optical imaging method without use of contrast agents.

Further methods have been described using light for detection of atherosclerotic plaque without use of any contrast agents. U.S. Pat. No. 5,275,594 (C. R. Bard) describes a method to distinguish between atherosclerotic plaque and normal tissue by analysing photoemissions from a target site. U.S. Pat. No. 5,197,470 (Eastman Kodak) describes a method and instrument using near IR to discriminate between healthy tissue and diseased tissue. The method might be used for diagnosis of plaque. U.S. Pat. No. 5,046,501 (Wayne State University) describes a method of identifying atherosclerotic plaque versus viable tissue using light with wavelength between 350 and 390 nm.

Methods directed to in vivo imaging using radiolabelled contrast agents have been described. See e.g. U.S. Pat. No. 5,976,496 (Diatide) describes labelled somatostatin analogs for imaging cardiovascular disease. The core of the invention is radio-labelled compounds. Fluorescent labelling is mentioned. U.S. Pat. No. 5,026,537 (Centocor) describes a method for imaging of atherosclerotic plaque using radio-labelled monoclonal antibodies that are specific for activated platelets or activated endothelial cells.

Vulnerable atherosclerotic plaque is still a challenge to diagnose and treat. There is a need for improved diagnostic methods, especially for diagnosis of vulnerable atherosclerotic plaque in an early stage with good reliability. We have surprisingly discovered that the use of optical imaging methods with new contrast agents fulfil these requirements.

### SUMMARY OF THE INVENTION

The present invention provides an optical imaging contrast agent with affinity for an abnormally expressed biological target associated with vulnerable atherosclerotic plaque.

The invention is also described in the claims.

The following definitions will be used throughout the document:

Vulnerable atherosclerotic plaque tissue: A deposit in the wall of a blood vessel that may become unstable and susceptible to rupture or fissure, thus precipitating thrombosis, particularly an acute coronary condition. Factors that contribute to risk of rupture include an inflamed, thin or fissured cap, and a large lipid core. Plaques at risk of erosive thrombosis commonly have an irregular or denuded inflamed lumen.

Abnormally expressed target: A target that is either overexpressed or downregulated in vulnerable atherosclerotic plaque tissue.

Overexpressed target: A receptor, an enzyme or another molecule or chemical entity that is present in a higher amount in vulnerable atherosclerotic plaque tissue than in normal tissue.

Downregulated target: A receptor, an enzyme or another molecule or chemical entity that is present in a lower amount in diseased tissue than in normal tissue.

### DETAILED DESCRIPTION OF THE INVENTION

A first aspect of the present invention is an optical imaging contrast agent for imaging of vulnerable atherosclerotic plaque. By the term optical imaging contrast, or just contrast agent, we mean a molecular moiety used for enhancement of image contrast in vivo comprising at least one moiety that interacts with light in the ultraviolet, visible or near-infrared part of the electromagnetic spectrum.

The contrast agent has affinity for an abnormally expressed target associated with vulnerable atherosclerotic plaque.

Vulnerable atherosclerotic plaque tissue containing a downregulated target can be identified by a low amount of bound imaging agent compared to normal tissue. In this situation, the amount of imaging agent should be less than 50% of that in normal tissue, preferably less than 10%.

Preferred contrast agents according to the invention, have affinity for an overexpressed target associated with vulnerable atherosclerotic plaque. Preferred targets are those targets that are more than 50% more abundant in vulnerable atherosclerotic plaque tissue than in surrounding tissue. More preferred targets are those targets that are more than two times more abundant in vulnerable atherosclerotic plaque tissue than in surrounding tissue. The most preferred targets are those targets that are more than 5 times more abundant in vulnerable atherosclerotic plaque tissue than in surrounding tissue.

Relevant groups of targets are receptors, enzymes, nucleic acids, proteins, lipids, other macromolecules as, for example, lipoproteins and glycoproteins. The targets may be located in the vascular system, in the extracellular space, associated with cell membranes or located intracellularly.

Preferred groups of targets are adhesion molecules, extracellular matrix proteins and glycans, hormones, cytokines and complement components, receptors, components of signal-transducing pathways and viruses associated with vulnerable atherosclerotic plaque.

The following biological targets are abnormally expressed in vulnerable atherosclerotic plaque tissue and are preferred targets for optical imaging contrast agents of the invention:

Adhesion Molecules

Alpha L (antigen CD11A (p180)), E-selectin, galectin-3, ICAM-1, beta 5 integrins, alpha 4 integrins, αᵥβ₃ and αᵥβ₅ integrins, Kistrin, MDC15, P-selectin, β1 integrins, VCAM-1, VE-cadherin, VLA-4, αMβ2 (CD11b/cd18) integrin.

Extracellular Matrix Proteins and Glycans

Aggrecan, biglycan, collagens (particularly Types I, II, III and IV), COMP, decorin, elastin, fibrillin, fibrin, fibrin degradation products, fibrin fragment E1, fibrinogen, fibromodulin, fibronectin, hyaluronan, osteopontin, perlecan, PRELP, Tenascin-C, versican, vitronectin.

Enzymes and Inhibitors

α₂macroglobulin, Arginase, type II,, CPP-32 (cysteine protease), cytosolic acyl coenzyme A thioester hydrolase, Lipase (lysosomal acid), mannosyl (alpha-1,6-)-glycoprotein beta-1,6-N-acetylglucosaminyltransferase, Nitric oxide synthase, PAPP-A, phospholipase 2, superoxide dismutase, extracellular, ubiquitin-conjugating enzyme E2L 3, Acyl-CoA-cholesterol acyltransferase, angiotensin-converting enzyme (ACE), cathepsin B, cathepsin D, cathepsin G, cathepsin K, cathepsin L, cathepsin S, collagenases, cyclooxygenase, inducible nitric oxide synthase (iNOS), matrix metalloproteinase such as MMP-1, MMP-3, MMP-8, MMP-9 (stromelysin), MMP-13, MMP-14, MDCs alias ADAMs, myeloperoxidase, prothrombin, sphingomyelinase, Tissue-type plasminogen activator, urokinase, Cystatin C, Tissue inhibitor of MMPs.

Hormones

Angiotensin II, endothelin, IGF-I, Inositol 1,4,5-triphosphate receptor, type 3, PDGF, TGF-β, Vascular endothelial growth factor (VEGF).

Immune System: Cytokines, Complement Componenets Etc.

Alpha M (complement component receptor 3), CCL11 (eotaxin), CCL17, CCL22, CD154, CD40, CD40L=CD154, Colony stimulating factor 3, Complement component 2, C-reactive protein, CX3CL1, CXCL10, endothelial monocyte-activating polypeptide, GMCSF, IFN-γ, IL-1, IL-10, IL-18, IL-18 binding protein, IL-1β,IL-2, IL-6, IL-8, MCP-1, MCSF, Small inducible cytokine subfamily A (Cys-Cys), member 18, Small inducible cytokine subfamily A (Cys-Cys), member 20, TNF-α.

Receptors

Activin A receptor type II-like 1, angiotensin II receptors, cannabinoid receptor 2 (macrophage), CCR-2, CD31, CD32 alias FcγIIR, CD36, CD4, CD44, CD68 (macrosialin), CD8, chemokine (C-C motif) receptor 5, chemokine-like receptor 1, colony stimulating factor 1 receptor, colony stimulating factor 2 receptor, colony stimulating factor 2 receptor-Beta, low-affinity (granulocyte macrophage), colony stimulating factor 2 receptor: Alpha, low-affinity (granulocyte macrophage), CX3CR1, CXCR2, CXCR3, endocytic receptor (macrophage mannose receptor family), folate receptor, G protein-coupled receptors, IL-10 receptor, IL-18 receptor, IL 8 receptors (CXCR1), interleukin 1 receptor, type I, interleukin 17 receptor, Interleukin 3 receptor, alpha (low affinity), Interleukin 8 receptor, beta, LOX, LR 11, LRP (LDL-receptor-like protein), LTB4 receptor, macrophage stimulating 1 receptor (c-met-related tyrosine kinase), MCSF receptor, MD2, MyD88, SR-A, SR-B1, SR-PSOX, TGF-β receptor, toll-like receptor 1, toll-like receptor 2, toll-like receptor 4, urokinase receptor (uPAR), VEGF receptor, VLDL receptor, T-cell receptor, alpha (V, D, J, C).

Signal-Transducing and Related Molecules

Actin-related protein 2/3 complex, subunit 1A (41 kD), adaptor-related protein complex 3, beta 2 subunit, adenylate cyclase 7, adenylate cyclase 8 (brain), aryl hydrocarbon receptor, calmodulin 2 (phosphorylase kinase, delta), calponin 1, basic, calponin 3, acidic, eukaryotic translation initiation factor 3, subunit 6 (48 kD), H3 histone family 3B (H3.3B), heterotrimeric G proteins, histone acetyltransferase 1, huntingtin-interacting protein A, hypoxia-inducible factor, interferon regulatory factor 5, kinesin-like 1, MAP kinases, mitogen-activated protein kinase kinase 4, mitogen-activated protein kinase kinase kinase 5, nuclear factor of activated T cells, cytoplasmic 1, PPAR-α, PPAR-γ, protein tyrosine phosphatase, receptor type, RAB33A, member RAS oncogene family, ribosomal protein L21, Serine/threonine protein-kinase, β-actin, tyrosine kinases, Uracil-DNA glycosylase, Zinc finger protein 272.

Viruses

Cytomegalovirus, epstein-Barr virus, hepatitis A virus, herpes simplex 1 & 2, HIV virus, influenza virus.

Others

ABCA1, adducts of nonenal and other oxidation products, adipophilin, AGEs (Advanced Glycation End Products), antigen identified by monoclonal antibody Ki-67, antithrombin II, ATPase, H+ transporting, lysosomal, Beta 2 (antigen CD18 (p95)), chlamydial heat shock protein 60, Coagulation factor XII (Hageman factor), dystrophin, EGF-containing fibulin-like extracellular matrix protein 1, epithelial V-like antigen 1, Factor XIII, GP IIb/IIIa, HBP/Vigilin, HSP20, HSP27, HSP-40 (HDJ-2), HSP60, HSP65, HSP70, Oxidized LDL, perilipin, phosphatidylserine, plakophilin 1, plasminogen activator inhibitor, secretory granule, neuroendocrine protein 1 (7B2 protein), Sialophorin (gpL115, leukosialin, CD43), β2-glycoprotein I, tissue factor pathway inhibitor 2, vasculin, Von Willebrand factor.

Among the more preferred targets for contrast agents for optical imaging of vulnerable atherosclerotic plaque are: Kistrin, collagens (particularly Types I, III and IV), cathepsin B, cathepsin K, matrix metalloproteinase 3 (stromelysin), matrix metalloproteinase 9, myeloperoxidase, urokinase, endothelin, angiotensin II, CCR-2, C-reactive protein, angiotensin II receptors, CD36, CD40, folate receptor, SR-A, SR-B1, Toll-like receptor 4, uPAR, VEGF receptor, LOX-1, PPAR-γ, Factor XIII, HBP/Vigilin, perilipin.

The most preferred targets for contrast agents for optical imaging of vulnerable atherosclerotic plaque are: matrix metalloproteinase 9, Toll-like receptors, scavenger receptors, oxidized LDL, oxidation products of lipids and their adducts with protein, angiotensin II receptors and collagens

Generally, any targets that have been identified as possible targets for agents for treatment of vulnerable atherosclerotic plaque are potential targets also in optical imaging.

The preferred contrast agents of the present invention are molecules with relatively low molecular weights. The molecular weight of preferred contrast agents is below 14 000 Daltons, preferably below 10000 Daltons and more preferably below 7000 Daltons.

The contrast agents are preferably comprised of a vector that has affinity for an abnormally expressed target in vulnerable atherosclerotic plaque tissue, and an optical reporter.

Thus viewed from one aspect the present invention provides a contrast agent of formula I:

V−L−R (I)

wherein V is one or more vector moieties having affinity for one or more abnormally expressed target in vulnerable atherosclerotic plaque tissue, L is a linker moiety or a bond and R is one or more reporter moieties detectable in optical imaging.

The vector has the ability to direct the contrast agent to a region of vulnerable atherosclerotic plaque. The vector has affinity for the abnormally expressed target and preferably binds to the target. The reporter must be detectable in an optical imaging procedure and the linker must couple vector to reporter, at least until the reporter has been delivered to the region of vulnerable atherosclerotic plaque and preferably until the imaging procedure has been completed.

The vector can generally be any type of molecules that have affinity for the abnormally expressed target. The molecules should be physiologically acceptable and should preferably have an acceptable degree of stability. The vector is preferably selected from the following group of compounds: peptides, peptoid/peptidomimetics, oligonucleotides, oligosaccharides, lipid-related compounds like fatty acids, traditional organic drug-like small molecules, synthetic or semi-synthetic, and derivatives and mimetics thereof. When the target is an enzyme the vector may comprise an inhibitor of the enzyme or an enzyme substrate. The vector of the contrast agent preferably has a molecular weight of less than 10 000 Daltons, more preferably less than 4500 Daltons and most preferably less than 2500 Daltons, and hence does not include antibodies or internal image antibodies. In addition to problems with immune reactions, long circulation time and limited distribution volume, many antibodies have an affinity for the receptor that is too low for use in imaging.

An optical imaging contrast agent comprising a vector having affinity for any of the preferred targets is a preferred embodiment of the invention.

Contrast agents having affinity for more than one abnormally expressed target related to the disease is an aspect of the invention. Such contrast agents can comprise two or more different vectors or molecular subunits that target two or more different abnormally expressed targets.

Another possibility according to the present invention is that the contrast agent comprises one vector that is able to bind to more than one abnormally expressed target in vulnerable atherosclerotic plaque.

A contrast agent according to the present invention can also comprise more than one vector of same chemical composition that bind to the abnormally expressed biological target.

Some receptors are unique to endothelial cells and surrounding tissues. Examples of such receptors include growth factor receptors such as VEGF and adhesion receptors such as the integrin family of receptors. Peptides comprising the sequence arginine-glycine-aspartic acid (RGD) are known to bind to a range of integrin receptors. Such RGD-type peptides constitute one group of vectors for targets associated with vulnerable atherosclerotic plaque.

Below are some examples of vectors having affinity for targets associated with vulnerable atherosclerotic plaque:

Vectors for Matrix Metalloproteinases:

Peptide sequence: Cys-Gly-Pro-Leu-Gly-Leu-Leu-Ala-Arg-OH

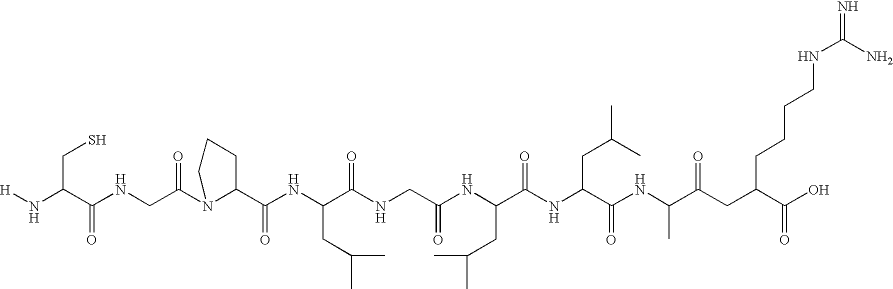

Vectors for Mapping of Tyrosine Kinase Activity of the Epidermal Growth Factor Receptor (EGFR):

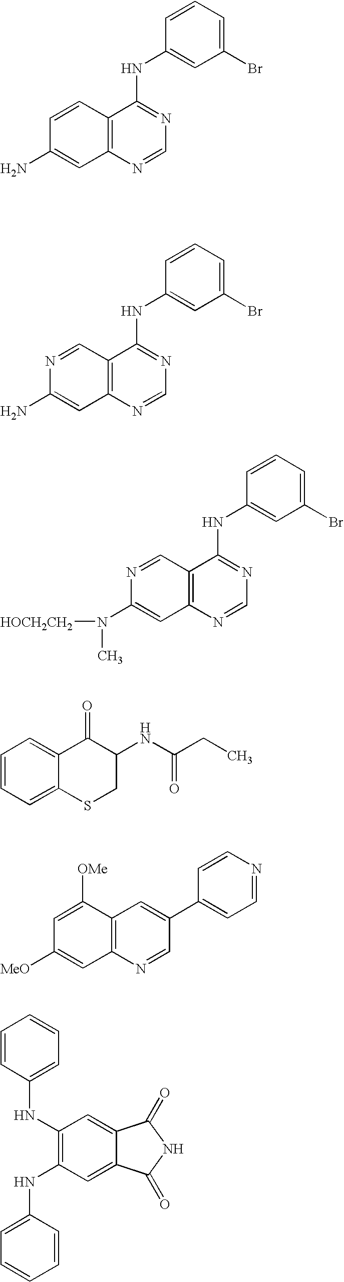

Gefitinib (Iressa®):

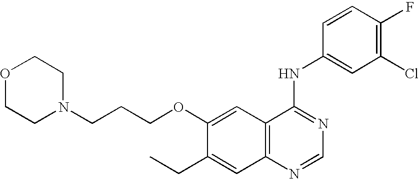

These represent a group of kinase inhibitors and are analogues of ATP.

Vector for Urokinase:

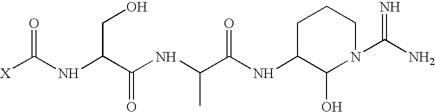

X=substituted sulfonic acid amid or alkoxy

The vector is a peptide derivative of an arginine aldehyde. The vector has been described by Tamura et al. (2000) in Bioorganic & Medicinal chemistry Letters 10 (9) 983-7.

Vector for Binding to Oxidated Phospholipids (Hydrazine Derivative):

H₂N—NH₂

Vector for Angiotensin:

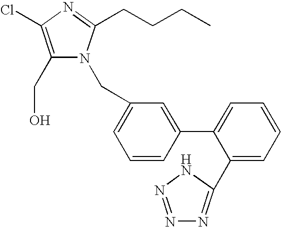

A wide variety of linkers can be used. The linker component of the contrast agent is at its simplest a bond between the vector and the reporter moieties. In this aspect the reporter part of the molecule is directly bound to the vector that binds to the abnormally expressed target. More generally, however, the linker will provide a mono- or multi-molecular skeleton covalenty or non-covalently linking one or more vectors to one or more reporters, e.g. a linear, cyclic, branched or reticulate molecular skeleton, or a molecular aggregate, with in-built or pendant groups which bind covalently or non-covalently, e.g. coordinatively, with the vector and reporter moieties. The linker group can be relatively large in order to build into the contrast agent optimal size or optimal shape or simply to improve the binding characteristics for the contrast agent to the abnormally expressed target in vulnerable atherosclerotic plaque tissue.

Thus, linking of a reporter unit to a desired vector may be achieved by covalent or non-covalent means, usually involving interaction with one or more functional groups located on the reporter and/or vector. Examples of chemically reactive functional groups which may be employed for this purpose include amino, hydroxyl, sulfhydroxyl, carboxyl and carbonyl groups, as well as carbohydrate groups, vicinal diols, thioethers, 2-aminoalcohols, 2-aminothiols, guanidinyl, imidazolyl and phenolic groups.

The reporter is any moiety capable of detection either directly or indirectly in an optical imaging procedure. The reporter might be a light scatterer (e.g. a coloured or uncoloured particle), a light absorber or a light emitter. More preferably the reporter is a dye such as a chromophore or a fluorescent compound. The dye part of the contrast agent can be any dye that interacts with light in the electromagnetic spectrum with wavelengths from the ultraviolet light to the near-infrared. Preferably, the contrast agent of the invention has fluorescent properties.

Preferred organic dye reporters include groups having an extensive delocalized electron system, eg. cyanines, merocyanines, indocyanines, phthalocyanines, naphthalocyanines, triphenylmethines, porphyrins, pyrilium dyes, thiapyriliup dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes. Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots).

Particular examples of chromophores which may be used include fluorescein, sulforhodamine 101 (Texas Red), rhodamine B, rhodamine 6G, rhodamine 19, indocyanine green, Cy2, Cy3, Cy3B, Cy3.5, Cy5, Cy5.5, Cy7, Cy7.5, Marina Blue, Pacific Blue, Oregon Green 488, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750. The cyanine dyes are particularly preferred.

Particularly preferred are dyes which have absorption maxima in the visible or near-infrared region, between 400 nm and 3 μm, particularly between 600 and 1300 nm.

The contrast agents can comprise more than one dye molecular sub-unit. These dye sub-units might be similar or different from a chemical point of view. Preferred contrast agents have less than 6 dye molecular sub-units.

Several relevant targets for vulnerable atherosclerotic plaque are enzymes. A contrast agent for optical imaging of vulnerable atherosclerotic plaque for targeting an enzyme can be an enzyme contrast agent substrate that can be transformed to a contrast agent product possessing different pharmacokinetic and/or pharmacodynamic properties from the contrast agent substrate. This embodiment of the invention provides contrast agent substrates having affinity for an abnormally expressed enzyme, wherein the contrast agent substrate changes pharmacodynamic and/or pharmacokinetic properties upon a chemical modification into a contrast agent product in a specific enzymatic transformation, and thereby enabling detection of areas of disease upon a deviation in the enzyme activity from the normal. Typical differences in pharmacodynamic and/or pharmacokinetic properties can be binding properties to specific tissue, membrane penetration properties, protein binding and solubility properties.

Alternatively, if the abnormally expressed target for diagnosis of vulnerable atherosclerotic plaque is an enzyme, the contrast agent for optical imaging can be a dye molecule that directly binds to the enzyme. The contrast agent will have affinity for the abnormally expressed enzyme, and this may be used to identify tissue or cells with increased enzymatic activity.

In a further aspect of the invention, the contrast agent changes dye characteristics as a result of an enzymatic transformation. For example, a fluorescent dye reporter of the contrast agent is quenched (no fluorescence) by associated quencher groups, until an enzymatic cleavage takes place, separating the dye from the quencher groups and resulting in fluorescence at the site of the abnormally expressed enzyme.

Another aspect of this part of the invention is that the dye may change colour, as e.g. a change in absorption and/or emission spectrum, as a result of an enzymatic transformation.

If the abnormally expressed target for diagnosis of vulnerable atherosclerotic plaque is a receptor or another non-catalytic target, the contrast agent for optical imaging can bind directly to the target and normally not change the dye characteristics.

The preferred contrast agents of the present invention are soluble in water. This means that the preferred contrast agents have a solubility in water at pH 7.4 of at least 1 mg/ml.

The contrast agents of the present invention can be identified by random screening, for example by testing of affinity for abnormally expressed targets of a library of dye labelled compounds either prepared and tested as single compounds or by preparation and testing of a mixture of compounds (a combinatorial approach). Alternatively, random screening may be used to identify suitable vectors, before labelling with a reporter.

The contrast agents of the present invention can also be identified by use of technology within the field of intelligent drug design. One way to perform this is to use computer-based techniques (molecular modelling or other forms of computer-aided drug design) or use of knowledge about natural and exogenous ligands (vectors) for the abnormally expressed targets. The sources for exogenous ligands can for example, be the chemical structures of therapeutic molecules for targeting the same target. One typical approach here will be to bind the dye chemical sub-unit (reporter) to the targeting vector so that the binding properties of the vector are not reduced.

This can be performed by linking the dye at the far end away from the pharmacophore centre (the active targeting part of the molecule).

The contrast agents of the invention are preferably not endogenous substances alone. Some endogenous substances, for instance estrogen, have certain fluorescent properties in themselves, but they are not likely to be sufficient for use in optical imaging. Endogenous substances combined with an optical reporter however, fall within the contrast agents of the invention.

The contrast agent of the invention are intended for use in optical imaging. Any method that forms an image for diagnosis of disease, follow up of disease development or for follow up of disease treatment based on interaction with light in the electromagnetic spectrum from ultraviolet to near-infrared radiation falls within the term optical imaging. Optical imaging includes all methods from direct visualization without use of any device and use of devices such as various scopes, catheters and optical imaging equipment, for example computer based hardware for tomographic presentations. The contrast agents will be useful with optical imaging modalities and measurement techniques including, but not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarisation, luminescence, fluorescence lifetime, quantum yield, and quenching.

Examples of contrast agent for optical imaging of vulnerable atherosclerotic plaque according to the invention are shown below:

Contrast Agent for Mapping of Matrix Metalloproteinase

The peptide vector (Cys-Gly-Pro-Leu-Gly-Leu-Leu-Ala-Arg) is linked to e.g. fluorescein (R) through a linker (L):

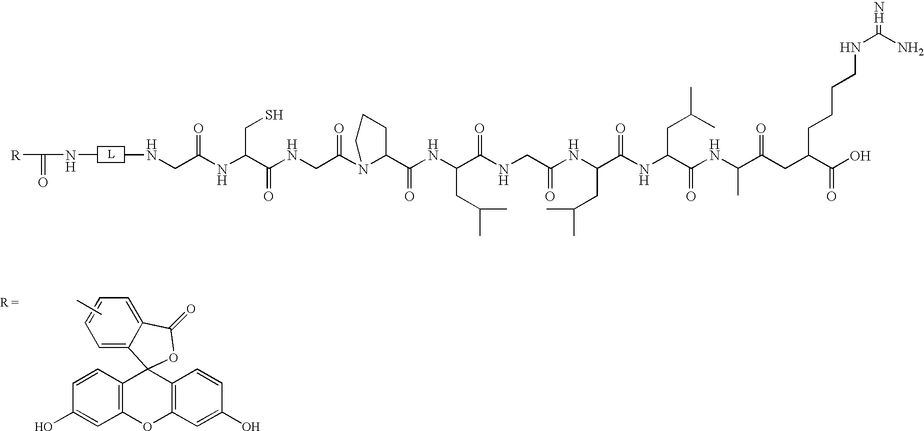

A suggested synthesis is given in example 1.

Contrast Agents for Mapping of Tyrosine Kinase Activity of the Epidermal Growth Factor Receptor (EGFR):

A suggested synthesis is given for preparation of a contrast agent comprising a vector with affinity for tyrosine kinase of the epidermal growth factor linked to a Cy5.5 reporter.

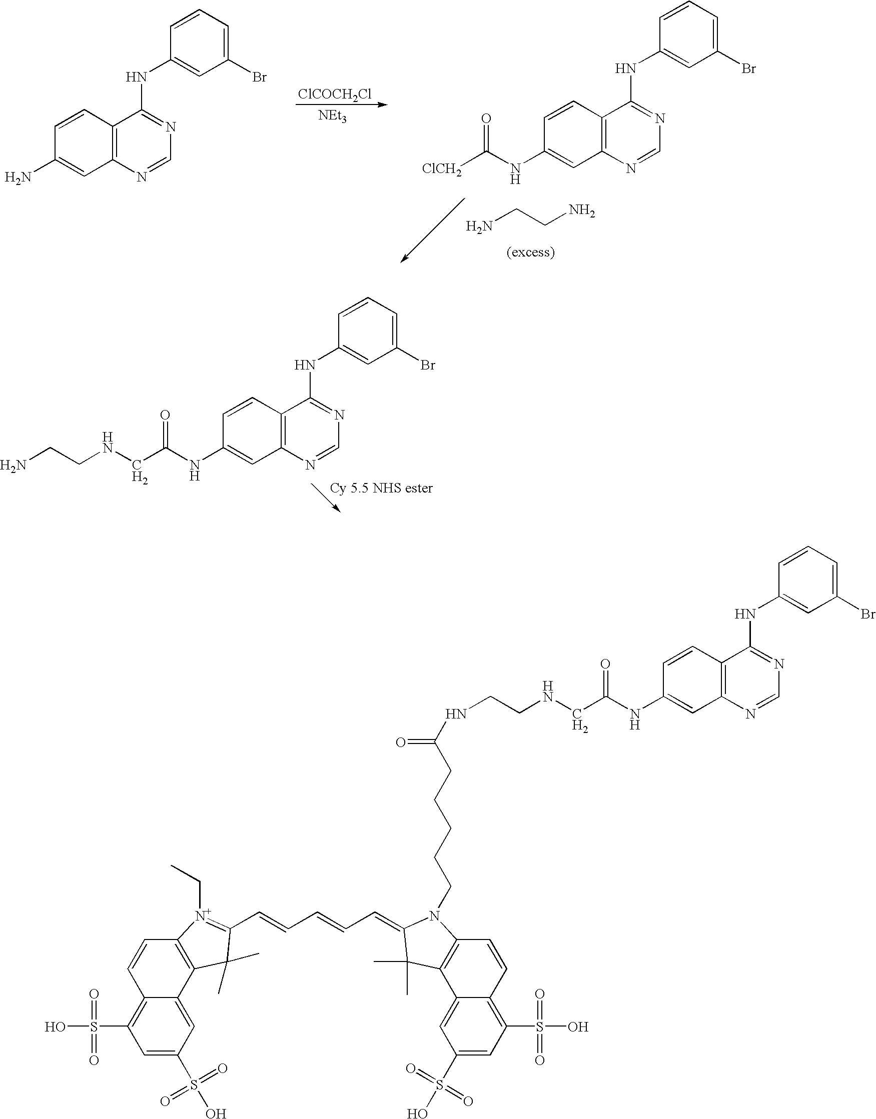

Contrast Agents With Affinity for Urokinase:

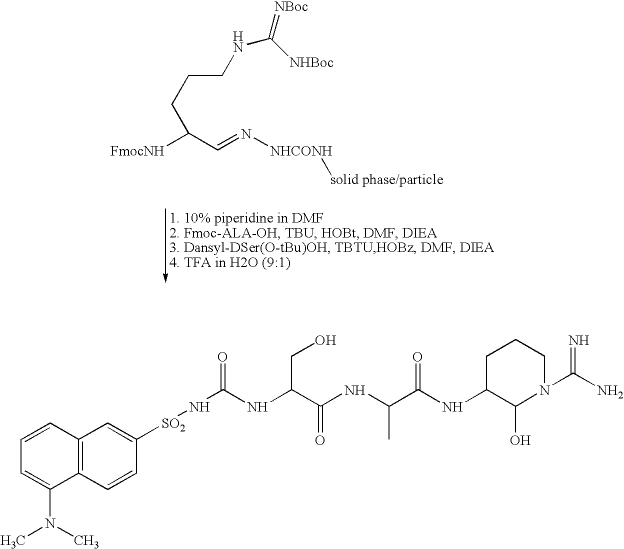

The solid phase conjugate is prepared according to S. Y. Tamura et al in Bioorganic & Medicinal Chemistry Letters 10 (2000) 983-98

Contrast Agent With Affinity for Oxidized Phospholipids:

The contrast agent comprises a fluorescein derivative conjugated with hydrazine.

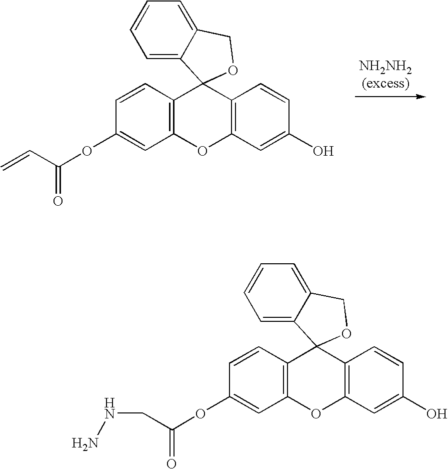

A further embodiment is the use of contrast agents of the invention for optical imaging of vulnerable atherosclerotic plaque, that is for diagnosis of vulnerable atherosclerotic plaque, for use in follow up the progress in vulnerable atherosclerotic plaque development, for follow up the treatment of vulnerable atherosclerotic plaque and fo surgical guidance.

In the context of this invention, diagnosis includes screening of selected populations, early detection, biopsy guidance, characterisation, staging and grading. Follow up of treatment includes therapy efficacy monitoring and long-term follow-up of relapse. Surgical guidance includes tumour margin identification during resection.

Still another embodiment of the invention is a method of optical imaging of vulnerable atherosclerotic plaque using the contrast agents as described.

Still another embodiment of the invention is a method of optical imaging for diagnosis of vulnerable atherosclerotic plaque, to follow up the progress of vulnerable atherosclerotic plaque development and to follow up the treatment of vulnerable atherosclerotic plaque.

One aspect of these methods is to administer the present contrast agents and follow the accumulation and elimination directly visually during surgery. Another aspect of these methods is to administer the present contrast agents and perform visual diagnosis through a fibre optic catheter. Alternatively, imaging of superficial major blood vessels, such as the carotid artery, can be performed non-invasively.

Still another aspect of the present invention is to administer the present contrast agents and perform the image diagnosis using computerized equipment as for example a tomograph.

Still another embodiment of the invention is use of a contrast agent as described for the manufacture of a diagnostic agent for use in a method of optical imaging of vulnerable atherosclerotic plaque involving administration of said diagnostic agent to an animate subject and generation of an image of at least part of said body.

Still another embodiment of the invention is pharmaceutical compositions comprising one or more contrast agents as described or pharmaceutically acceptable salts thereof for optical imaging for diagnosis of vulnerable atherosclerotic plaque, for follow up progress of vulnerable atherosclerotic plaque development or for follow up the treatment of vulnerable atherosclerotic plaque. The diagnostic agents of the present invention may be formulated in conventional pharmaceutical or veterinary parenteral administration forms, e.g. suspensions, dispersions, etc., for example in an aqueous vehicle such as water for injections. Such compositions may further contain pharmaceutically acceptable diluents and excipients and formulation aids, for example stabilizers, antioxidants, osmolality adjusting agents, buffers, pH adjusting agents, etc. The most preferred formulation is a sterile solution for intravascular administration or for direct injection into area of interest. Where the agent is formulated in a ready-to-use form for parenteral administration, the carrier medium is preferably isotonic or somewhat hypertonic.

The dosage of the contrast agent of the invention will depend upon the clinical indication, choice of contrast agent and method of administration. In general, however dosages will be between 1 micro gram and 70 grams and more preferably between 10 micro grams and 5 grams for an adult human.

The present invention is particularly suitable for methods involving parenteral administration of the contrast agent, e.g. into the vasculature or directly into an organ or muscle tissue, intravenous administration being especially preferred.

The following examples are illustrative only and not intended to be limiting. Other features and advantages of the invention will be apparent from the detailed description and from the claims.

### EXAMPLES

### Example 1

### Contrast Agent for Mapping of Matrix Metalloproteinase (MMP). Synthesis of Fluorescein-Cys-Gly-Pro-Leu-Gly-Leu-Leu-Ala-Arg-OH Linker Conjugate

Step 1

The peptide component was synthesised on an ABI 433A automatic peptide synthesiser starting with Fmoc-Arg(Pmc)-wang resin on a 0.1 mmol scale using 1 mmol amino acid cartridges. The amino acids were pre-activated using HBTU before coupling. An aliquot of the peptide resin was then transferred to a clean round bottom flask an N-methyl morpholine (1 mmol) in DMF (5 ml) added followed by chloroacetyl chloride (1 mmol). The mixture was gently shaken until Kaiser test negative. The resin was extensively washed with DMF.

Step 2

5(6)-carboxyfluorescein (188 mg, 0.5 mmol) and dicyclohexylcarbodiimide (113 mg, 0.55 mmol) are dissolved in DMF (20 ml). The mixture is stirred for 2 hours and cooled to 0° C. A solution of hexamethylenediamide (116 mg, 1 mmol) and DMAP (30 mg) in DMF is added and the mixture is stirred at ambient temperature for 72 hours. The solution is evaporated and the conjugate between carboxyfluorescein and hexamethylene-amine is isolated as monoamide by chromatography (silica, chloroform and methanol).

Step 3

The resin from step 1 is suspended in DMF (5 ml) and amide-amine conjugate from step 2 (0.5 mmol) pre-dissolved in DMF (5ml) containing triethylamine (0.5 mmol) is added. The mixture is heated to 50° C. for 16 hours then excess reagents filtered off, following extensive washing with DMF, DCM and diethyl ether then air drying. The product is treated with TFA containing TIS (5%), H₂O (5%), and phenol (2.5%) for 2 hours.

Excess TFA is removed in vacuo and the peptide is precipitated by the addition of diethyl ether. The crude peptide conjugate is purified by preparative HPLC C C-18, acetonitril, TFA, water).

### Example 2

### Contrast Agent for Mapping of Tyrosine Kinase Activity of the Epidermal Growth Factor.

Step 1. 4-[(3-bromophenyl)amino]-7-[N-(2-hydroxy-ethyl)-N-methylamino] pyrido [4,3-d] pyrimidine is prepared according to A. M. Thomson et al in J. Med. Chem. (1997) 40 3915-3925.

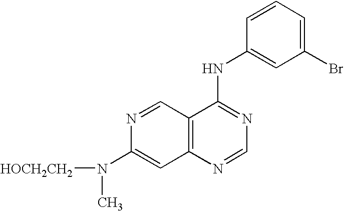

Step 2. 5(6)-carboxyfluorescein (1 mmol), dicyclohexylcarbodiimide (1.2 mmol) and DMAP (50 mg) are dissolved in DMF (30 ml). The mixture is stirred for 24 hours. A solution of the alcohol from step 1 (1 mmol) in DMF (5 ml) is added and the mixture is stirred for 3 days at ambient temperature. The fluorescein ester conjugate with the alcohol vector is isolated by chromatography (silica, hexane/chloroform).

### Example 3

### Contrast Agent with Affinity for Oxidized Phospholipids

Fluorescein-o-acrylate (1 mmol) and hydrazine hydrate (10 mmol) are dissolved in toluene (50 ml). The mixture is stirred for 24 hours at 100° C. The mixture is evaporated and the fluorescein hydrazine conjugate is isolated by flash chromatography using silica and methanol/chloroform/hexane.

### Example 4

### Contrast Agent for Urokinase

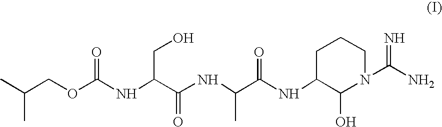

The ligand (I) is prepared according to S. Y. Tamura et al in Bioorganic & Medicinal Chemistry Letters 10 (2000) 983-987.

The ligand (I) (1 mmol) is dissolved in DMF. CY7-NHS ester (1 mmol) is added. The mixture is stirred for 5 days. The solvent is evaporated and the Cy-7-conjugate isolated by flash chromatography (silica, hexane, ethyl acetate).

### Example 5

### Contrast Agent with Affinity for Integrins: RGD Peptide Linked to Cy5.5

Step 1. Assembly of Amino Acids

The peptide sequence Asp-D-Phe-Lys-Arg-Gly was assembled on an Applied Biosystems 433A peptide synthesizer starting with 0.25 mmol Fmoc-Gly-SASRIN resin. An excess of 1 mmol pre-activated amino acids (using HBTU; O-Benzotriazol-1-yl-N,N,N′,N′-tetramethyluronium hexafluorophosohate) was applied in the coupling steps. The cleavage of the fully protected peptide from the resins was carried out by treatment of the resin with three portions of 35 mL of 1% trifluoroacetic acid (TFA) in dichloromethane (DCM) for 5 minutes each. The filtrates containing the peptide was immediately neutralised with 2% piperidine in DCM. The organics were extracted with water (3×100 mL), dried with MgSO₄ and evaporated in vacuo. Diethyl ether was added to the residue and the precipitate washed with ether and air-dried affording 30 mg of crude protected peptide. The product was analysed by analytical HPLC (conditions: Gradient, 20-70% B over 10 min where A=H₂0/0.1% TFA and B=CH₃CN/0.1% TFA; flow, 2 mL/min; column, Phenomenex Luna 3μ 5×4.6 mm; detection, UV 214 nm; product retention time 7.58 min). Further product characterisation was carried out using electrospray mass spectrometry (MH⁺ calculated, 1044.5; MH⁺ found, 1044.4).

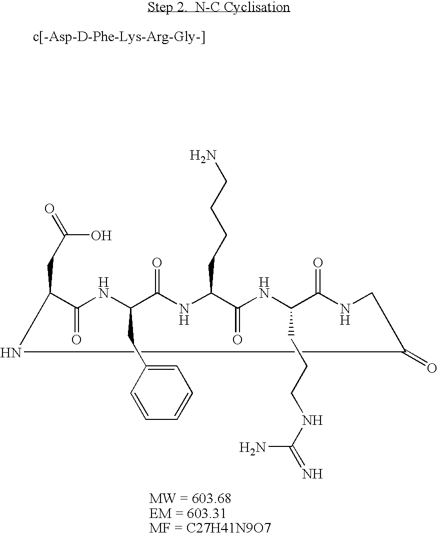

30 mg of the fully protected peptide, 16 mg of PyAOP, 4 mg of HOAt and 6 μL of N-methylmorpholine (NMM) were dissolved in dimethylformamide/DCM (1:1) and stirred over night. The mixture was evaporated in vacuo and diethyl ether added to the residue. The precipitate was washed with ether and air-dried. The crude cyclic fully protected peptide was treated with a solution of 25 mL TFA containing 5% water, 5% triisopropylsilane and 2.5% phenol for two hours. TFA was evaporated in vacuo and diethyl ether added to the residue. The precipitate was washed with ether and air-dried. Purification by preparative RP-HPLC (0-30% B over 40 min, where A=H₂O/0.1% TFA and B=CH₃CN/0.1% TFA, at a flow rate of 10 mL/min on a Phenomenex Luna 5μ C18 250×21.20 mm column) of the crude material afforded 2.3 mg pure product peptide. The pure product was analysed by analytical HPLC (conditions: Gradient, 0-15% B over 10 min where A=H₂O/0.1% TFA and B=CH₃CN/0.1% TFA; flow, 2 mL/min; column, Phenomenex Luna 3μ 5×4.6 mm; detection, UV 214 nm; product retention time 6.97 min). Further product characterisation was carried out using electrospray mass spectrometry (MH⁺ calculated, 604.3; MH⁺ found, 604.4).

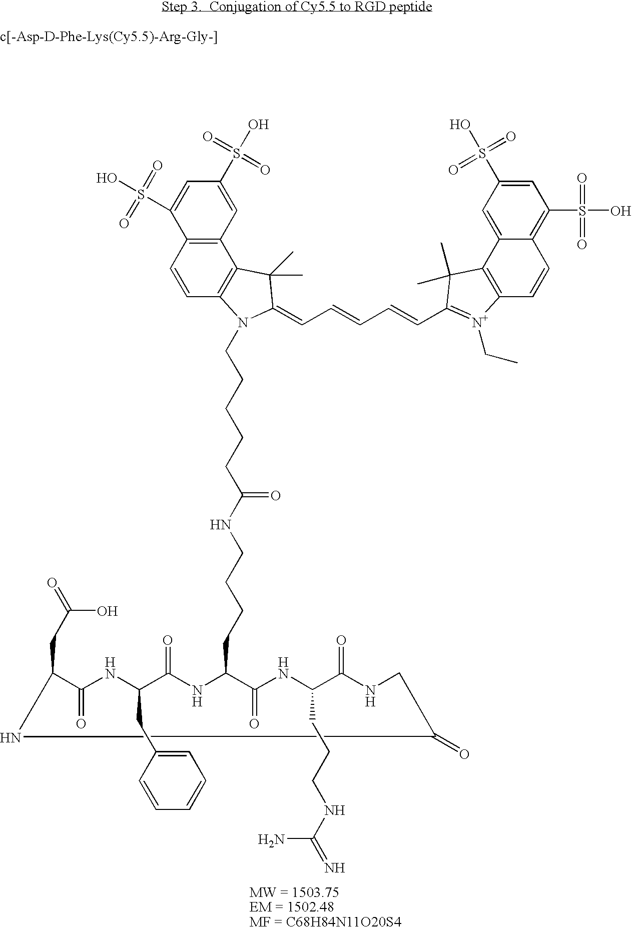

0.6 mg of the RGD peptide, 1.7 mg of Cy5.5 mono NHS ester and 5 μL of NMM were dissolved in 1 mL of dimethylformamide (DMF) and the reaction mixture stirred for 2 hrs. Diethyl ether was added to the DMF solution and the blue precipitate washed with diethyl ether and air-dried affording 0.7 mg of crude RGD peptide conjugated to Cy5.5.The pure product was analysed by analytical HPLC (conditions: Gradient, 5-50% B over 10 min where A=H₂O/0.1% TFA and B=CH₃CN/0.1% TFA; flow, 0.3 mL/min; column, Phenomenex Luna 3μ 5×2 mm; detection, UV 214 nm; product retention time 8.32 min). Further product characterisation was carried out using electrospray mass spectrometry (MH⁺ calculated, 1502.5; MH⁺ found, 1502.6).

### Example 6

### Synthesis of 3-[(4′-Fluorobiphenyl-4-sulfonyl)-(1-hydroxycarbamoylcyclopentyl)amino]propionic Acid (Compound A) Derivatised with Cy5.5—Contrast Agent for Binding to MMP

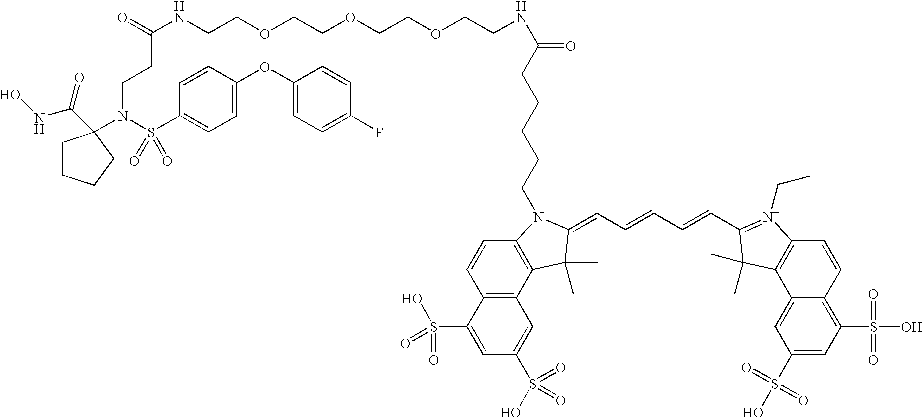

a) 1,11-Diazido-3,6,9-trioxaundecane

A solution of dry tetraethylene glycol (19.4 g, 0.100 mol) and methanesulphonyl chloride (25.2 g, 0.220 mol) in dry THF (100 ml) was kept under argon and cooled to 0° C. in an ice/water bath. To the flask was added a solution of triethylamine (22.6 g, 0.220 mol) in dry THF (25 ml) dropwise over 45 min. After 1 hr the cooling bath was removed and stirring was continued for 4 hrs. Water (60 ml) was added. To the mixture was added sodium hydrogencarbonate (6 g, to pH 8) and sodium azide (14.3 g, 0.220 mmol), in that order. THF was removed by distillation and the aqueous solution was refluxed for 24 h (two layers formed). The mixture was cooled and ether (100 ml) was added. The aqueous phase was saturated with sodium chloride. The phases were separated and the aqueous phase was extracted with ether (4×50 ml). Combined organic phases were washed with brine (2×50 ml) and dried (MgSO₄). Filtration and concentration gave 22.1 g (91%) of yellow oil. The product was used in the next step without further purification.

b) 11-Azido-3,6,9-trioxaundecanamine

To a mechanically, vigorously stirred suspension of 1,11-diazido-3,6,9-trioxaundecane (20.8 g, 0.085 mol) in 5% hydrochloric acid (200 ml) was added a solution of triphenylphosphine (19.9 g, 0.073 mol) in ether (150 ml) over 3 hrs at room temperature. The reaction mixture was stirred for additional 24 hrs. The phases were separated and the aqueous phase was extracted with dichloromethane (3×40 ml). The aqueous phase was cooled in an ice/water bath and pH was adjusted to ca 12 by addition of KOH. The product was extracted into dichloromethane (5×50 ml). Combined organic phases were dried (MgSO₄). Filtration and evaporation gave 14.0 g (88%) of yellow oil. Analysis by MALDI-TOF mass spectroscopy (matrix: □-cyano-4-hydroxycinnamic acid) gave a M+H peak at 219 as expected. Further characterisation using ¹H (500 MHz) and ¹³C (125 MHz) NMR spectroscopy verified the structure.

c) Linking Compound A to PEG(4)-N₃

To a solution of compound A (CP-471358, Pfizer, 41 mg, 87 μmol) in DMF (5 ml) were added 11-azido-3,6,9-trioxaundecanamine (19 mg, 87 μmol), HATU (Applied Biosystems, 33 mg, 87 μmol) and DIEA (Fluka, 30 μl, 174 μmol). After one hour reaction time the mixture was concentrated and the residue was purified by preparative HPLC (column Phenomenex Luna C18(2) 5 μm 21.2×250 mm, solvents: A=water/0.1% TFA and B=acetonitrile/0.1% TFA; gradient 30-60% B over 60 min; flow 10.0 ml/min, UV detection at 214 nm), giving 33.9 mg (59%) of product after lyophilisation. LC-MS analysis (column Phenomenex Luna C18(2) 3 μm 50×4.60 mm, solvents: A=water/0.1% TFA and B=acetonitrile/0.1% TFA; gradient 20-100% B over 10 min; flow 1 ml/min, UV detection at 214 nm, ESI-MS) gave a peak at 4.88 min with m/z 667.4 (MH⁺) as expected.

d) Synthesis of Compound A-PEG(4)-NH₂

To a solution of the PEG(4)-N₃ compound from c) (4.7 mg, 7.0 μmol) in methanol (4 ml) was added Pd/C (Koch-Light, ca 10 mg) added. The mixture was stirred at room temperature under hydrogen atmosphere (1 atm) for 10 min. The mixture was filtered and concentrated. LC-MS analysis (column Phenomenex Luna C18(2) 3 μm 50×4.60 mm, solvents: A=water/0.1% TFA and B=acetonitrile/0.1% TFA; gradient 20-100% B over 10 min; flow 1 ml/min, UV detection at 214 nm, ESI-MS) gave a peak at 4.17 min with m/z 641.4 (MH⁺) as expected. The product was used directly in the next step without further purification.

e) Conjugation of Cy 5.5

To a solution of the amine from d) (1.0 mg, 1.5 μmol) in DMF (0.2 ml) was added Cy 5.5-NHS (Amersham Biosciences, 1.0 mg, 1.0 μmol) and N-methylmorpholine (1 μl, 9 μmol). The reaction mixture was stirred for 48 h. MS analysis of the solution gave a spectrum showing starting material and the conjugated product at m/z 1539.7 (M⁺⁾, expected 1539.4.

### Example 7

### Cy5-VEGF

Five micrograms of vascular endothelial growth factor (VEGF-121, cat.no. 298-VS/CF) (carrier-free, from R&D Systems) were dissolved in 19 μl of 0.02 M borate buffer, pH 8.5. To this solution was added 2.5 nmol of the N-hydroxysuccinimide ester of a carboxylic acid derivative of Cy5 (Amersham Biosciences), dissolved in 5 μl of the same buffer. The reaction mixture was incubated for one hour in the dark at room temperature. Unreacted dye was separated from the fluorescent protein derivative by centrifuging through a Micro-Spin 6 gel filtration colum (Bio-Rad, exclusion limit about 6 kDa). The eluate fluoresced with excitation light at 646 nm, the emission being measured at 678 nm. The product was a fluorescent targeting molecule for the VEGF receptor.

### Example 8

### Cy5-TIMP-1

Five micrograms of tissue inhibitor of metalloproteinases-1 (TIMP-1, cat.no. 970-TM) (carrier-free, from R&D Systems) were dissolved in 25 μl of 0.02 M borate buffer, pH 8.5. To this solution was added 2.5 nmol of the N-hydroxysuccinimide ester of a carboxylic acid derivative of Cy5 (Amersham Biosciences), dissolved in 5 μl of the same buffer. The reaction mixture was incubated for one hour in the dark at room temperature. Unreacted dye was separated from the fluorescent protein derivative by centrifuging through a Micro-Spin 6 gel filtration column (Bio-Rad, exclusion limit about 6 kDa). The eluate fluoresced with excitation light at 646 nm, the emission being measured at 678 nm. The product was a fluorescent targeting molecule for matrix metalloproteinases.

### Example 9

### Fluorescein-TIMP-1

Five micrograms of tissue inhibitor of metalloproteinases-1 (TIMP-1, cat.no. 970-TM) (carrier-free, from R&D Systems) were dissolved in 25 μl of 0.02 M borate buffer, pH 8.5. To this solution was added 2.5 nmol of the N-hydroxysuccinimide ester of a carboxylic acid derivative of fluorescein (Fluka), dissolved in 5 μl of the same buffer. The reaction mixture was incubated for one hour in the dark at room temperature. Unreacted dye was separated from the fluorescent protein derivative by centrifuging through a Micro-Spin 6 gel filtration column (Bio-Rad, exclusion limit about 6 kDa). The eluate fluoresced with excitation light at 485 nm, the emission being measured at 538 nm. The product was a fluorescent targeting molecule for matrix metalloproteinases.

What is claimed is:

1. An optical imaging contrast agent with affinity for an abnormally expressed biological target associated with vulnerable atherosclerotic plaque.

2. A contrast agent as claimed in claim 1 wherein the molecular weight is below 14 000 Daltons.

3. A contrast agent as claimed in claim 1 of formula I
V-L-R, (I)
wherein V is one or more vector moieties having affinity for an abnormally expressed target in vulnerable atherosclerotic plaque, L is a linker moiety or a bond and R is one ore more reporter moieties detectable in optical imaging.

4. A contrast agent as claimed in claim 1 comprising a contrast agent substrate, wherein the target is an abnormally expressed enzyme, such that the contrast agent changes pharmacodynamic properties and/or pharmacokinetic properties upon a chemical modification from a contrast agent substrate to a contrast agent product upon a specific enzymatic transformation.

5. A contrast agent as claimed in claim 1 having affinity for any of the targets selected from kistrin, collagens, cathepsin B, cathepsin K, matrix metalloproteinase 3, matrix metalloproteinase 9, myeloperoxidase, urokinase, endothelin, CCR-2, C-reactive protein, angiotensin II receptors, CD36, CD40, folate receptor, SR-A, SR-B 1, Toll-like receptor 4, uPAR, VEGF receptor, LOX-1, PPAR-γ, Factor XIII, HBP/Vigilin, perilipin.

6. A contrast agent as claimed in claim 3 wherein V is selected from peptides, peptoid moieties, oligonucleotides, oligosaccharides, fat-related compounds, and traditional organic drug-like small molecules.

7. A contrast agent as claimed in claim 3 wherein R is a dye that interacts with light in the wavelength region from the ultraviolet to the near-infrared part of the electromagnetic spectrum.

8. A pharmaceutical composition for optical imaging of vulnerable atherosclerotic plaque comprising a contrast agent as defined in claim 1 together with at least one pharmaceutically acceptable carrier or excipient.

9. The contrast agent as claimed in claim 1 wherein the contrast agent is used for the manufacture of a diagnostic agent.

10. A method of optical imaging of vulnerable atherosclerotic plaque of an animate subject involving administering a contrast agent as defined in claim 1 to the subject and generating an optical image of at least a part of the subject to which said contrast agent has distributed.

11. The method as claimed in claim 10 for diagnosis of vulnerable atherosclerotic plaque, for follow up of the progress of vulnerable atherosclerotic plaque development, for follow up of treatment of vulnerable atherosclerotic plaque or for surgical guidance.

12. The contrast agent as defined in claim 1 wherein the contrast agent is used for optical imaging of vulnerable atherosclerotic plaque.

13. (canceled)


SEQUENCE LISTING (2 sequences)

SEQ ID NO: 1 - protein, 5 aa, Artificial sequence
  Synthetic peptide
DFKRG

SEQ ID NO: 2 - protein, 9 aa, Artificial Sequence
  Synthetic peptide
CGPLGLLAR